(19) United States
(12) Patent Application Publication
(10) Pub. No.: US 2009/0098263 A1
(43) Pub. Date: Apr. 16, 2009

(54) Device And Method For Rotary Fluidized Bed In A Succession Of Cylindrical Chambers

(76) Inventor: Axel de Broqueville, Grez-Doiceau (BE)

Correspondence Address: FINA TECHNOLOGY INC, PO BOX 674412, HOUSTON, TX, 77267-4412, US

(21) Appl. No.: 11/793,094

(22) PCT Filed: Dec. 9, 2005
(86) PCT No.: PCT/EP05/56639

§ 371 Date: Mar. 25, 2008

(30) Foreign Application Priority Data
Dec. 15, 2004 (BE) 2004/0612

(51) Int. Cl. A23L 1/025; B01J 8/20; B01J 8/28; C07C 4/06; C08F 2/01; G05D 3/00

(52) U.S. Cl. 426/465; 422/140; 422/142; 422/105; 526/88; 585/439

(57) ABSTRACT

The invention concerns a device and a method for rotary fluidized bed for catalytic polymerization, drying and other treatments of solid particles or for catalytic transformation of fluids, wherein a cylindrical reactor (1), in which the fluids are injected (7) tangentially to its cylindrical wall, is divided into a succession of cylindrical chambers (Z1, Z2, Z3) by hollow discs (3), which are fixed to its cylindrical wall, which have central openings through which the fluids circulating in rotation inside the cylindrical chambers are sucked (10), which have lateral openings through which said fluids are evacuated through the cylindrical wall of the reactor and which have passages (27) for transferring the suspended solid particles in the rotary fluidized bed from one chamber to the next through said discs (3).

The present invention relates to a device with a rotating fluidized bed in a succession of cylindrical chambers for the catalytic polymerization, drying, impregnation, or other treatments of solid particles, in suspension in said rotating fluidized beds, passing from one chamber to the other, by a fluid or fluid mixture, or for the cracking, dehydrogenation or other catalytic conversions of a fluid or fluid mixture, passing through the rotating fluidized beds, composed of solid catalytic particles passing from one cylindrical chamber to the other.

Methods in which solid particles are in suspension in a fluid and thereby form a fluidized bed which is traversed by this fluid, are well known. When this fluid is injected tangentially to the side wall of a cylindrical reactor, it can transfer part of its kinetic energy to the solid particles to give them a rotational movement, and if the energy transferred is sufficient, this rotational movement produces a centrifugal force which can keep the solid particles along the reactor wall thereby forming a rotating fluidized bed, whereof the surface is approximately an inverted truncated cone, if the reactor is vertical. Such a method is the subject of Belgian patent application No. 2004/0186, filed Apr. 14, 2004, in the name of the same inventor.

However, when a fluid jet is injected at high velocity into a large reactor, it is rapidly slowed down by its expansion, depending on the conditions under which it is injected. This is why, when the density of the fluid is much lower than that of the particles, it is necessary to have a very high flow rate in order to transfer momentum to the solid particles producing a sufficient centrifugal force, and the devices for removing this fluid, after it has passed through the fluidized bed, may become bulky and limit the height or length of the reactor.

In the present invention, a cylindrical reactor is divided into a succession of cylindrical chambers by a succession of flat cylinders or hollow disks fixed against its side wall. These hollow disks comprise openings at their center to suck in the fluid passing through each chamber and rotating rapidly, and openings in their side wall to remove it from the reactor. These hollow disks are perforated with appropriately profiled passages to enable the solid particles in suspension in the fluid, rotating rapidly, to pass from one cylindrical chamber to the other.

In the present invention, the fluid or fluid mixture is injected tangentially along the cylindrical wall of the reactor, generally as thin films, and, while rotating, radially traverses the reactor, from its side wall to its center, from where it is removed via the central openings in the hollow disks. The injection velocity of the fluid and its flow rate are sufficient to make the solid particles in suspension rotate in a rotating fluidized bed with a rotational velocity that produces a centrifugal force which separates them from the central openings of the hollow disks whereby the fluid is removed, and enabling their transfer from one cylindrical chamber to the other, via passages in the hollow disks, despite any slight pressure difference between these cylindrical chambers.

In the present invention, the fluid is fed by one or more distributors, outside the reactor, in order to distribute them appropriately to the injectors located in the various cylindrical chambers. It is then removed, via hollow disks, by one or more fans or compressors, which suck it through one or more collectors, outside the reactor and interconnected, in order to regularize the pressures in the various cylindrical chambers. The fluid can then be recycled, after suitable treatment, for example cooled or heated, by the same distributors or other distributors, to the same or the next cylindrical chambers. It can be recycled several times to the same cylindrical chambers or to successive cylindrical chambers.

The solid particles are generally introduced at one end of the reactor and then transferred from one cylindrical chamber to the other, thanks to their rotational velocity and the profile of the passages through the hollow disks. They are generally removed at the opposite end of the reactor. A solid particle recycle device may be provided outside the reactor.

The present invention, to improve the efficiency of energy transfer between the fluid and the solid particles, may comprise deflectors suitably profiled and arranged close to the fluid injectors, to permit the mixing of the fluid with a limited quantity of solid particles and to channel the fluid to prevent or reduce its expansion in the reactor before it has transferred a substantial quantity of its kinetic energy to these solid particles. This device is suitable for using fluids which are much lighter than the solid particles and for injecting it at high velocity into a large reactor without losing a large part of its kinetic energy due to its expansion in the reactor. Such a device is described in the Belgian patent application in the name of the same inventor filed on the same day as the present application.

The present invention may comprise sets of helical turns or transverse fins, inclined or wound in a spiral and fixed along the cylindrical wall of the cylindrical chambers, to use part of the rotational kinetic energy of the solid particles to make them rise along this wall, in order to reduce the difference in thickness between the top and bottom of the fluidized bed. This device makes it possible to increase the height of the cylindrical chambers without having to increase the thickness of the fluidized bed at its base. Such a device is described in Belgian patent application No. 2004/0186, filed Apr. 14, 2004 in the name of the same inventor.

The reactor may be horizontal. In this case, the velocity with which the fluid injected into the reactor and its flow rate must be sufficient to make the fluidized bed rotate with a rotational velocity that produces a sufficient centrifugal force for its thickness in the upper part of the reactor to be close to its thickness in the lower part of the reactor, and the openings normally provided in the center of the hollow disks may be slightly offset downward to center them better with regard to the approximately cylindrical surface of the fluidized bed.

This method serves to increase the difference in velocity between the solid particles and the fluid without reducing the density of the fluidized bed thanks to the centrifugal force, and hence to improve the contact and heat transfer between them. It also serves to significantly increase the volume of fluid passing through the fluidized bed and thereby to significantly reduce the fluid residence time in the fluidized bed.

The division of the reactor into a succession of cylindrical chambers, which may be interconnected only by small passages, serving to transfer solid particles accompanied by a small quantity of fluid, makes it possible to have them traversed by different fluids, recycled in a loop. This makes the method particularly advantageous if it is necessary to use fluids with compositions significantly varying from one cylindrical chamber to the other.

This method permits particle residence times in the reactor, which are short or long according to the dimensions of the passages between the cylindrical chambers, and the resistance to the rotation of the fluidized bed may be low, because the injection of the fluid as thin films along the side wall of the reactor reduces the friction of the solid particles against this wall.

This method is particularly advantageous when the volume of fluid flowing is very high, because the devices for central removal of the fluid by the hollow disks can permit very high fluid flow rates with a minimum of resistance and, since the fluid distributors and collectors are outside the reactor, they may have large diameters without reducing the space available for the fluidized bed inside the reactor.

This method is also particularly advantageous when the pressure in the reactor is lower than atmospheric pressure, because the hollow disks can support the cylindrical wall of the reactor, thereby making it possible to have thin walls, cut longitudinally, to form slits through which the fluid can be injected, or to facilitate disassembly. Furthermore, the distributors, collectors and reactor can form an easily transportable compact assembly.

Thus, this method permits the construction of lightweight, compact, transportable and efficient units, for example for drying cereal grains. It is also suitable for catalytic modifications of fluids at low pressure, like the cracking of light olefins or the dehydrogenation of ethylbenzene which, being highly endothermic, require intermediate heating and catalyst regeneration. It can also be used for catalytic, bimodal or multimodal copolymerization of particles in suspension in a succession of active fluids having different compositions.

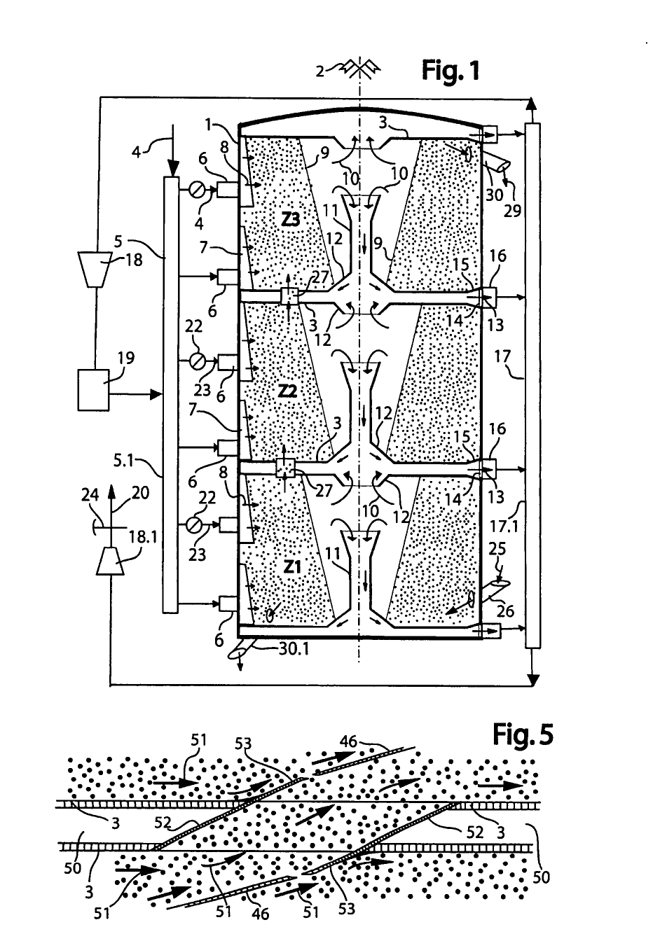

FIG. 1 shows a schematic view of a cross section of a vertical cylindrical reactor whereof the cross section of its cylindrical side wall (1) on each side of its cylindrical axis of symmetry (2) can be seen. A succession of hollow disks whereof the hollow sections (3) are visible, divides the reactor into a succession of cylindrical chambers or zones, from Z1 to Z3. The fluid (4) is fed by the distributor (5) to the sets of tubes (6), distributed around the reactor and connected to sets of injectors (7) distributed inside the reactor and designed to inject the fluid, generally as thin films, horizontally and tangentially to the reactor wall, that is, perpendicular to the plane of the figure. While rotating, the fluid passes through the fluidized bed which contains solid particles in suspension, symbolized by black dots. It approaches the center of the reactor at a radial velocity, symbolized by the arrows (8), which is of an order of magnitude lower than its rotational velocity. After having crossed the approximately conical surface of the fluidized bed, of which the cross section (9) is shown, the fluid (10) enters the central openings of the hollow disks (3), which may be surmounted by tubes (11) to prevent the solid particles from entering therein during shutdowns, and which may be enlarged (12) around their central openings to facilitate the entry of the fluid. The fluid (13) is then removed, via the openings (14) of the side edges of the hollow disks which may be enlarged (15) around these openings (14) to facilitate the outflow of the fluid, via sets of tubes (16) distributed around the reactor, toward a collector (17) connected to a fan or compressor (18), which sucks out the fluid to recycle it, after suitable treatment in (19), through the lower part (5.1) of the distributor, via a set of tubes (6) and injectors (7), distributed around the reactor and feeding the lower zones of the reactor. The fluid may be recycled several times before being removed at (20), through the lower part (17.1) of the collector, by the fan or compressor (18.1). The average number of fluid recyclings is approximately equal to the ratio of the capacities of the fans (18) and (18.1).

It should be observed that the fluid injection velocity is influenced by the hydrostatic pressure generated by the weight of the fluidized bed in each zone. To avoid an excessive difference in fluid injection velocity and flow rate between the base and the top of each zone, the slits (7) through which the fluid is injected can be profiled appropriately, as symbolized by their trapezoidal shape, and they can be equipped with obstacles appropriately distributed to reduce the injection velocity in their upper part. Control valves (22) can also be used to adjust the velocity and proportion of the fluid (23) injected at the various levels of the cylindrical chambers. A control valve (24) can also adjust the outlet flow rate of the fluid (20).

The solid particles (25) can be introduced into the bottom of the reactor via the tube (26) by appropriate means, such as gravity, a helical screw or a fluid jet. Since the reactor is divided by hollow disks into several cylindrical chambers, from Z1 to Z3, they rise from one chamber to the next, via passages (27) which are arranged through the hollow disks. They are removed from the last cylindrical chamber, Z3, at the top of the reactor, at (29), via the tube (30) by appropriate means. Other outlets, (30.1), can be provided, for example in the bottom of each chamber, to empty the reactor rapidly.

The quantity of particles transferred depends on the rotational velocity of these particles, which must be sufficient to overcome the hydrostatic pressure of the fluidized bed located above the passage. Thus by increasing the proportion and velocity of fluid injected at the top of a cylindrical chamber using a control valve (22), the energy injected into the top of this chamber is increased and thereby also the rotational velocity of the solid particles and hence their transfer to the upper zone. By servocontrolling these valves by detectors of the surface level of the fluidized beds of each chamber, these surfaces can be stabilized between the passages and the central inlet of the hollow disks. These passages can thereby be localized against the side wall of the reactor, where the particle concentration is the highest, and thereby reduce the quantity of fluid entrained with these solid particles.

The quantity of solid particles transferred from one zone to the other may also vary according to whether the passages are more or less immersed in the fluidized bed of the lower cylindrical chamber, which serves to stabilize the surface of the fluidized bed at the top of each cylindrical chamber along these passages. Thereby, at equilibrium, the fluidized bed may be more or less thick according to the distance of these passages from the side edge of the reactor.

The reactor can be drained via side outlets at the bottom of each zone and it can be filled initially from the bottom, by shutting off the fluid feed via the tubes (6) of the upper unfilled cylindrical chambers during the filling of a lower cylindrical chamber, to prevent the majority of the fluid from passing through the empty chambers. This can also be done through recycled fluid feed tubes, if the size and nature of the solid particles permit, or through the top, if permitted by the orientation of at least one passage per hollow disk.

The thin fluid film leaving the injectors tends to widen very rapidly and hence to slow down before having transferred sufficient rotational kinetic energy to the solid particles. To avoid this, appropriately profiled side deflectors can be fixed more or less parallel to the side wall of the reactor, close to the injector outlets, in order to mix a limited volume of solid particles with the fluid injected in the spaces or corridors located between these side deflectors and the reactor wall. These side deflectors prevent the expansion of the fluid, and hence its slowdown, before it has transferred a sufficient part of its kinetic energy to the solid particles, in these spaces or corridors, which must have a profile and a length appropriate to the objectives.

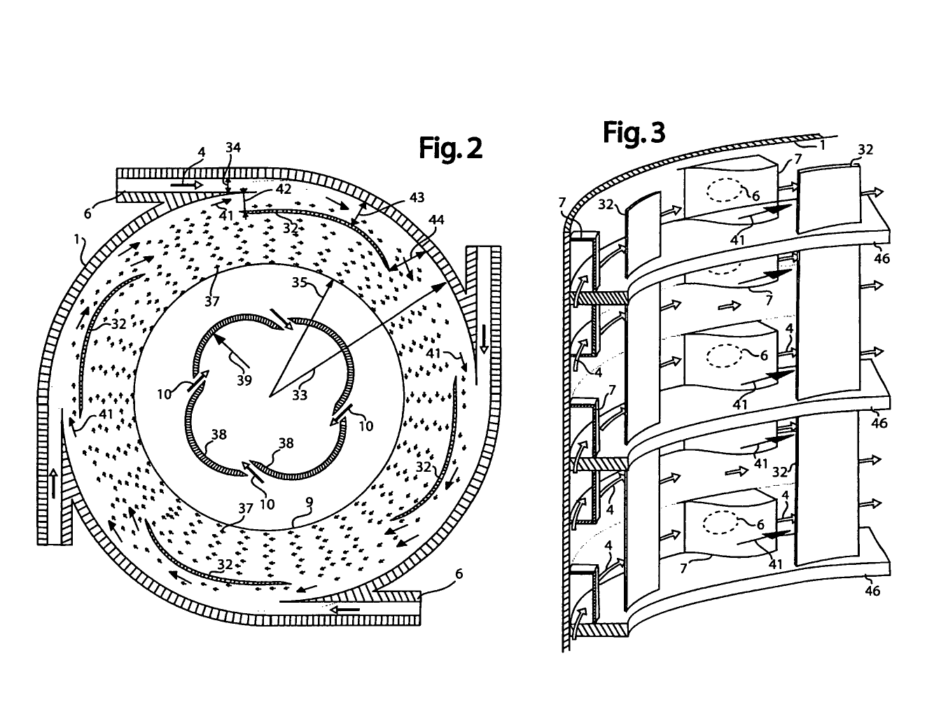

FIG. 2 shows a cross section of the reactor in order to show this fluid injection pattern. It shows the cross section of the side deflectors (32), perpendicular to the plane of the figure and extending along the cross sections of the side wall (1) of the reactor, of radius (33), in order to bound, with this side wall, a space or corridor, generally convergent then divergent, through which the fluid, illustrated by the arrows (4), injected via the tubes or nozzles (6), of width (34), must pass. It also shows the circular cross section of the surface of the fluidized bed (9) of radius (35). The solid particles are illustrated by small arrows (37), indicating their travel direction.

In this figure, the access tubes to the hollow disks, not shown, are connected by central deflectors, perpendicular to the plane of the figure, having cross section (38), curvature (39), bounding slits through which the fluid (10) is sucked into the central openings of the hollow disks, for better separation of the fluid from the particles.

Concentrated streams of solid particles, symbolized by the arrows (41), enter these spaces or corridors, generally convergent then divergent, via access passages or corridors, of width (42), located between the wall of the injectors (6) and the side deflectors (32), at a velocity which is about the average rotational velocity of the solid particles in the reactor. These concentrated streams of solid particles are diluted when mixing with the injected fluid, which yields a substantial part of its kinetic energy to them, thereby increasing their momentum, in these spaces or corridors between the reactor walls (1) and the side deflectors (32). The solid particles then mix with the other solid particles of the fluidized bed, yielding to them the momentum acquired.

In FIG. 2, these spaces or corridors are first convergent, to reach a minimum width (43), and then divergent, to reach an outlet width (44). They may also have a constant width. In this case, the fluid slows down as the solid particles and the fluid accompanying them accelerate. In general, the dimensions of these spaces or corridors must be defined according to the operating conditions and the kinetic energy transfer objectives.

It is also necessary to take account of the decrease in hydrostatic pressure of the fluidized bed, along the cylindrical surface of the reactor, as a function of the height in the cylindrical chambers of the reactor. The fluid leaving the injectors may tend to rise along the reactor walls before mixing with the solid particles, owing to this hydrostatic pressure difference along this wall. To avoid this, transverse deflectors, perpendicular to the cylindrical wall of the reactor, like rings for example, can divide the space bounded by the fins and the side wall of the reactor, to guide the fluid and the particles in the desired direction, generally horizontal or inclined upward, until the fluid is mixed with the particles, as shown in FIG. 3.

FIG. 3 is an axonometric projection of a piece of side wall (1) of the reactor, for better visualization of an example of fluid injectors (7), with their side deflectors (32) and rings (46), serving as transverse deflectors preventing the fluid from rising along the reactor wall. It also shows, by a dotted line, the inlets of the fluid feed tubes (6), located behind the side wall of the injectors, and, cross-hatched, the cross sections of the injector outlets (7) in the foreground. The arrows (4) and (41) respectively indicate the directions of the streams of fluid and solid particles entering or leaving the convergent and divergent spaces between the side deflectors (32) and the side wall (1) of the reactor.

The transverse deflectors, shown by wide rings (46), may be hollow, forming sorts of circular nozzles, and may be connected to the exterior of the reactor by one or more feed tubes in order to distribute the fluid to a succession of injectors placed along them, to reduce the number of tubes passing through the reactor wall, needed to feed the injectors, which may be desirable if the pressure in the reactor is high.

These transverse deflectors may also be successions of helical turns, forming an upward spiral, continuous or discontinuous, inside each cylindrical chamber, or they may be a succession of fractions of helical turns or transverse fins, grouped at the same or several of the same levels of the chambers, the upper edge of one fraction of turn or fin overhanging the lower edge of the next one, in order to make the solid particles rise along the reactor wall and thereby reduce the difference in thickness of the fluidized bed and the differences in pressure along this wall between the top and bottom of the various cylindrical chambers of the reactor.

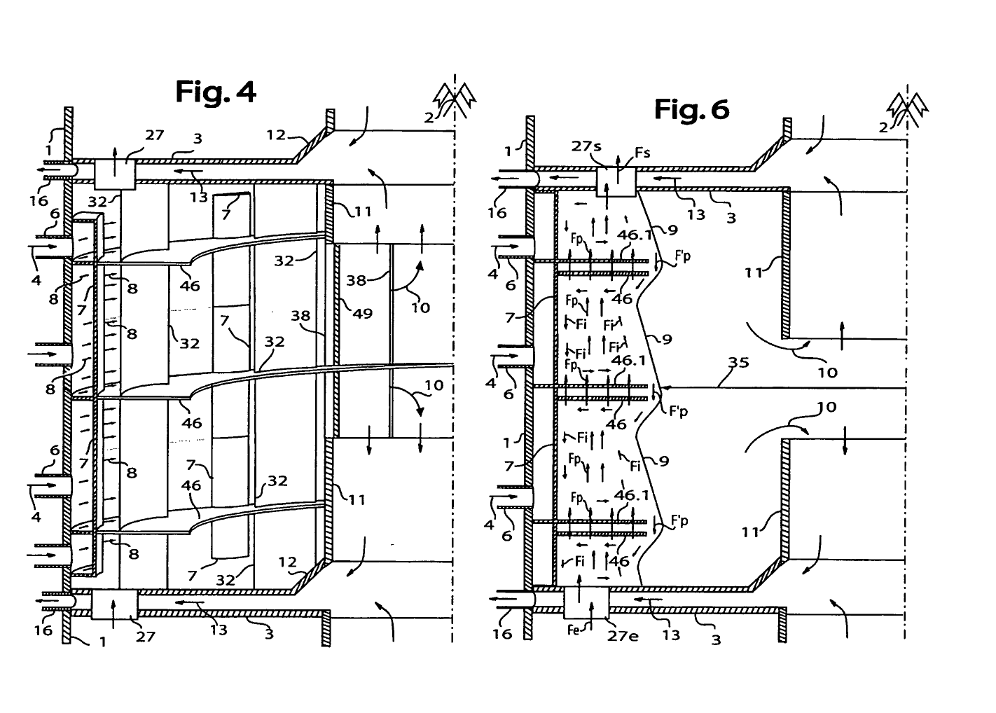

FIG. 4 is the projection of a half cross section of a cylindrical chamber, in which successions of quarters of helical turns (46) form either one continuous spiral making three turns inside the chamber, or three sets of four helical turns located at the same levels of the chamber and succeeding each other at 90° intervals, the upper edge of one quarter of a turn overhanging the lower edge of the next. The figure shows: the cross sections of the hollow disks (3), of the fluid (4) feed tubes (6), of the inlet tubes (11) of the hollows disks, widened at (12) and connected by central deflectors (38), whereof a cross section (49) can be seen; the arrows (8), (10) and (13) respectively symbolizing the outgoing fluid stream (8) from the injectors (7), the incoming fluid stream (10) in the central tubes (11) via the slits bounded by the central deflectors (38), and radially (13) passing through the hollow disks (3) toward the outlet tubes (16) of the reactor; the passages (27) for the transfer of particles from one zone to the other, the side deflectors (32), the fluid injectors (7) and their cross sections in the foreground, forming, from the bottom upward, continuous sets, separated by quarters of helical turns (46).

FIG. 5 shows the cross section of a passage (27). It shows the cross section (3) of the two parallel plates forming the hollow disk and its internal space (50) through which the fluid passes radially, that is, perpendicular to the plane of the figure, to leave the reactor. The solid particles are shown by black dots which shift in the direction of the arrows (51). They pass through the hollow disk running along the inclined walls (52) of the passage. They are prolonged by deflectors (53) on each side of the hollow disk to facilitate the transfer of the particles from the bottom upward, in the direction of their rotational velocity. These deflectors (53) may be prolonged by spirals of which the cross section (46) is shown, to facilitate the upward movement of the solid particles.

FIG. 6 is a transverse flow diagram of the solid particles along a half-longitudinal section of a cylindrical chamber similar to the one shown in FIG. 4, without the side and central deflectors. The cross section (1) of the reactor wall can be recognized, its cylindrical axis of symmetry (2), the fluid (4) feed tubes (6) in the injectors of cross section (7), sections (46) of the start of the quarters of helical turns along the side wall of the cylindrical chamber, located below the sections (46.1) of the end of the quarters of helical turns located in the quarter of the cylindrical chamber in the foreground of the figure.

The fluid (4), injected into the cylindrical chamber, perpendicular to the plane of the figure, passes through the surface of the fluidized bed of section (9) and enters (10) the inlet tubes (11) of the hollow disks (3), from which it is sucked out by the outlet tubes (16). The solid particles, of which the rotational velocity perpendicular to the plane of the figure is of an order of magnitude higher than the transverse velocities, enter the cylindrical chamber via the lower passage, (27e), at a flow rate Fe and exit therefrom via the upper passage (27s) at flow rate Fs. If the latter is higher than the inlet flow rate, Fe, the chamber empties progressively of its solid particles and the surface of the fluidized bed approaches its side wall, thereby automatically decreasing the outlet flow rate Fs. Another way to adjust the level of the fluidized bed is to servocontrol the injection flow rate of fluid in the upper part of the chamber to a particle detector, which may be placed along the lower wall of the hollow disk and which, depending on the position of the surface of the fluidized bed, increases or decreases this flow rate and thereby the rotational velocity of the solid particles and thereby the quantity of solid particles transferred via the passage (27s).

The solid particles, rotating in the fluidized bed inside the cylindrical chamber, are thrust upward by the sets of quarters of helical turns, at a flow rate Fp, symbolized by the upward arrows. If this flow rate is higher than the outlet flow rate, Fs, they must fall back into the space between the helical turns and the tubes (11), at a flow rate F′p=Fp−Fs, and the centrifugal force keeps them in the fluidized bed, whereof the surface is wavy around the helical turns. These turns, by supporting the weight of the fluidized bed located above them, undergo a pressure difference between their lower surface and their upper surface, which serves to decrease the pressure difference between the bottom and the top of the cylindrical chamber. They thereby serve to reduce the difference in thickness of the fluidized bed between the top and bottom of the cylindrical chamber, and thereby increase its height.

The difference in pressure between the top and bottom of the cylindrical chamber may cause differences in fluid injection velocity according to the height of their injection. These differences generate differences in rotational velocities of the solid particles. Moreover, the difference in pressure between the two sides of the hollow disks, and more particularly between the inlet and outlet of the passages through these hollow disks and the friction, slow down the solid particles which are transferred from one chamber to the other and thereby reduce the rotational velocity of the solid particles in the bottom of the next cylindrical chamber.

The slower rotational velocity of the solid particles and hence the lower centrifugal force in the bottom of the cylindrical chambers causes both a slight decrease in the pressure along the side wall and a slight increase in the thickness of the fluidized bed, thereby decreasing the slope of its surface which depends on the ratio of the centrifugal force to the force of gravity. These differences in pressure and slope generate an internal flow, which tends to reduce these differences and which is directed downward along the side wall, symbolized by the downward arrows, Fi, and upward close to the surface of the fluidized bed, symbolized by the upward arrows, Fi.

Similarly, the solid particles are slowed down by the friction and the increase in their potential energy while rising along the upper surface of the helical turns, thereby causing the same type of internal flow between the sets of helical turns. These successive reductions of the rotational velocity of the solid particles and their internal flow increase the quantity of energy that the fluid has to transfer to the particles, necessitating an efficient transfer of momentum and a very high fluid flow rate, appropriate for this method.

The internal flow can be estimated approximately by dividing the fluidized bed into rings whereof the average rotational velocities are assumed, and determining the pressure and thickness differences between these rings to determine the extent of this flow, and then applying the conservation of momentum to determine the average equilibrium rotational velocity of these rings by successive approximations.

These velocities depend, inter alia, on the momentum transferred by the fluid to the solid particles. In an open space, this momentum depends on the rotational velocity of the fluid, which is more closely related to the proportions of the cylindrical chamber and the fluid flow rate, than to its injection velocity. In contrast, the variation in pressure in a convergent space serves to transfer a momentum to the solid particles which is related to its kinetic energy and hence its injection velocity, thereby favoring this type of feed when the ratio of the fluid injection velocity to the desired rotational velocity of the solid particles must be very high because of the high ratio between the particle and fluid densities.

This device can be adapted to various patterns, according to the various methods.

### Method of Catalytic Polymerization of Solid Particles

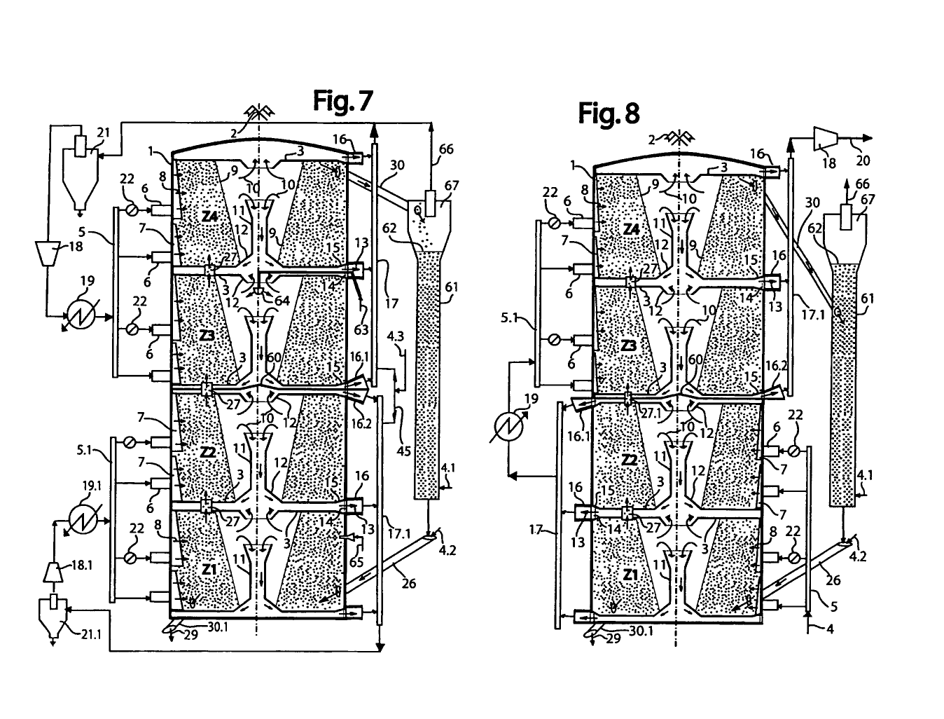

FIG. 7 shows a simplified diagram, similar to FIG. 1, slightly modified to permit the bimodal or multimodal polymerization of solid particles, serving as catalyst, in suspension in fluids or mixtures of active fluids, containing the monomer and the comonomer(s), such as, for example, the catalytic bimodal copolymerization of ethylene with hexene.

The figure shows the reactor (1), its cylindrical axis of symmetry (2), the hollow sections of the hollow disks (3) dividing the reactor into two sets of two successive cylindrical chambers, from Z1 to Z2 and Z3 to Z4, the feed tubes (6), with their control valves (22), the section of the injectors (7), the sections (9) of the surfaces of the fluidized beds, the inlet tubes (11) of the hollow disks and the outlet tubes (16).

There are two sets of independent distributors (5) and (5.1), two sets of collectors (17) and (17.1), connected together by a tube (45) to balance the pressures in the two sets of cylindrical chambers, two compressors (18) and (18.1), with their fluid treatment units, symbolized by the heat exchangers, (19) and (19.1), and the cyclones (21) and (21.1), and the hollow disk, separating the chamber Z2 from the chamber Z3, is divided by a separation partition (60) preventing the mixing of the fluids issuing from these two chambers, in order to recycle separately the fluids flowing in each of the sets of cylindrical chambers, from Z1 to Z2 and from Z3 to Z4. The number of sets of cylindrical chambers and the number of cylindrical chambers per set may vary. It depends on the size of the reactor and the polymerization objectives.

The polymer particles, symbolized by black dots, leaving the top of the reactor via the tube (30) are introduced into a recycle tube which may be a purification column (61), traversed by the fluid injected at (4.1), fluidizing the polymer particles which form a fluidized bed having a surface (62), the fluid exiting at (66) and passing through the particle separator (67) to be recycled by the compressor (18). The polymer particles are then recycled by the tube (26) at the bottom of the reactor. After having completed a certain number of cycles, they (29) are removed via tubes (30.1), which may be placed along the side walls of the various cylindrical chambers.

The feed of fresh monomer, such as ethylene, can be introduced: partly at (4.1), at the bottom of the purification column to be recycled to the upper part of the reactor after having purged the polymer particles of excess comonomer, such as hexene, which they contain; partly at (4.2), to facilitate the recycling of the polymer particles, although the hydrostatic pressure of the fluidized bed of the column (61), determined by its surface equilibrium level (62), may suffice; and partly in the pressure balancing tube (45), to prevent the pressure balancing between the upper set and the lower set of the cylindrical chambers from causing undesirable fluid transfers between these sets.

The comonomer (63), such as hexene, may be sprayed in fine droplets on the surface of the fluidized beds of one or more upper cylindrical chambers by injectors (64), which pass through the hollow disks, and the catalyst may be introduced by an appropriate device (65) in one of the cylindrical chambers. Other active components, such as hydrogen, and other monomers may be introduced into one of the recycle circuits, and their excess can be removed in the other recycle circuit, for example by absorption in regenerable absorbers. If necessary, additional inactive cooling fluids, such as propane or isobutane, can be sprayed in fine droplets on the fluidized beds in the same way as the comonomer.

This arrangement serves to limit undesirable fluid transfers from one set to the other, to the fluids not removed by the purification column (41) and to the fluids accompanying the polymer particles in the passage(s) (27) connecting the cylindrical chambers Z2 and Z3, whereof the size may be limited according to the polymerization objectives.

The control, purification accessories, etc., including the possibility of cooling the hollow disks, the purification column and other surfaces arranged in the chambers, are not described. They can be defined according to the polymerization objectives by the persons in charge of fluidized bed polymerization processes.

### Method of Catalytic Conversion of Fluids:

FIG. 8 shows a simplified diagram, similar to that of FIG. 7, slightly modified, for the catalytic conversion of a fluid or fluid mixture, in a rotating fluidized bed containing solid catalyst particles, such as, for example, catalytic cracking of light olefins.

In this arrangement, the fluid to be converted (4) is injected, preheated if necessary, into the distributor(s) (5) which feed the set of lower cylindrical chambers, Z1 and Z2. It is removed from these chambers by the collector(s) (17) to be heated in the heater (19), and recycled by the distributor(s) (5.1) to the set of upper cylindrical chambers, Z3 and Z4, wherefrom it is sucked out via the collector(s) (17.1) by a single compressor (18), to be transferred at (20) to appropriate treatment units.

The fresh or recycled catalyst powder is fed to the cylindrical chamber Z1 at the bottom of the reactor by the tube (26) and rises slowly from one chamber to the other, up to the top of the reactor, from where it is removed via the tubes (30), to a regeneration column (61). A regeneration fluid (4.1), for example a mixture of air and steam, fluidizes the catalyst powder in the regenerator while regenerating it. It is removed, at (66), through a particle separator (67). The equilibrium level of the surface (62) of the fluidized bed of the column (61) is the one giving a sufficient hydrostatic pressure for recycling the regenerated catalyst powder at the desired flow rate. This recycling may be facilitated by injecting a drive fluid (4.2), such as steam.

The feed in series of the two sets of cylindrical chambers creates a significant pressure difference between the chamber Z2 and the chamber Z3, thereby accelerating the catalyst particles and the fluid accompanying them in the passage (27) connecting them. This necessitates reducing the dimensions of this passage, which may be located at the distance from the side wall corresponding to the desired thickness of the fluidized bed, or which may be controlled by a flow control valve servocontrolled by level detectors of the fluidized bed of the cylindrical chamber Z2.

If the ratio of the density of the fluidized bed to that of the fluid is very high, not only is a very high fluid flow rate necessary, but also a high injection velocity, so that an appropriate device should be used for the transfer of energy and momentum from the fluid to the catalyst particles, before the fluid has lost a substantial part of its velocity owing to its expansion in the open space of the cylindrical chambers.

The number of chambers and sets may vary. The control, purification accessories, etc., are not described. They may be defined according to the objectives, by persons in charge of fluidized bed catalytic conversion processes.

In this arrangement, the outgoing fluid, issuing from the upper set of cylindrical chambers, is at lower pressure, which is generally favorable to the conversion of the fluid, but it is in contact with the catalyst which must be regenerated, which is unfavorable and requires shorter cycle times between two regenerations. This can be avoided by adding a second compressor before the heater (19) to equalize the pressures in the two sets of cylindrical chambers, thereby reversing the fluid flow, that is, to feed the fluid to be converted in the upper set and to remove the converted fluid from the lower set.

### Method of Drying or Other Treatments of Solid Particles:

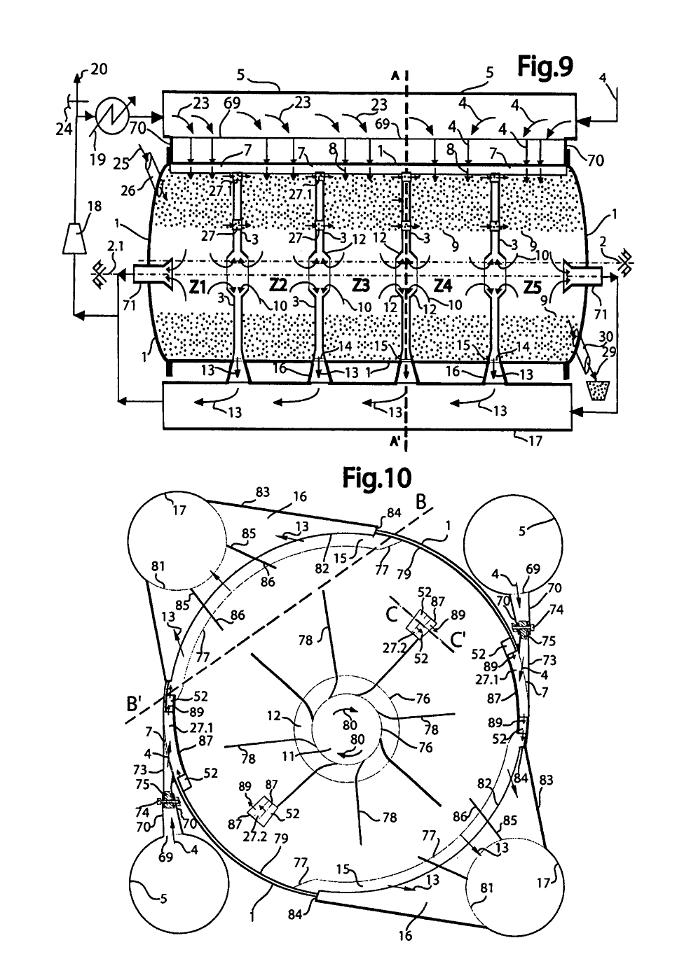

Solid particles, such as cereal grains, can be dried with air at a pressure close to atmospheric pressure, making it possible, thanks to this method, to implement lightweight, compact and easily transportable units, as described in FIGS. 9 to 12.

FIG. 9 shows the longitudinal cross section of a horizontal reactor, suitable for operating at a pressure slightly below atmospheric pressure. It shows the cross section (1) of its wall, its cylindrical axis of symmetry (2) and the hollow sections (3) of the hollow disks dividing the reactor into five successive cylindrical chambers, from Z1 to Z5. The distributor (5) is perforated with a longitudinal slit, symbolized by the thin line (69) and is connected by plates, replacing the tubes (6) and illustrated by the rectangle (70), with long longitudinal slits along the whole length of the reactor, symbolized by the rectangle (7), dividing the cylindrical wall of the reactor into two half-cylinders and designed to inject the fluid (4) perpendicular to the plane of the figure, that is tangentially in the reactor.

As it rotates, the fluid, at a radial velocity (8), passes through the fluidized bed, whereof the surface (9) is approximately cylindrical. However, the rotational velocity of the particles, symbolized by black dots, being higher in the lower part of the reactor owing to the force of gravity, the thickness of the fluidized bed is lower there and hence the axis of symmetry (2.1) of the surface of the fluidized bed is slightly lower than the axis of symmetry (2) of the reactor. The distance between these two axes, δ, which is about equal to half of the difference in thickness between the top and bottom of the fluidized bed, is approximately δ≡E·(2R−E)·g/2v², where E, R, g and v are respectively the average thickness of the fluidized bed, the radius of the cylindrical chambers, the gravitational acceleration, and the average rotational velocity of the solid particles, if R−E/2<<v²/g.

The fluid (10) then enters through the central openings of the hollow disks (3) widened (12) around said openings. The fluid (13) leaves the reactor via the openings (14), in thin lines, which are long transverse slits cut out of the side wall of the hollow disks which are widened (15) around the slits and it enters via the nozzles (16) into the collector with cross section (17) and is sucked out by a fan (18). Tubes (71), passing through the ends or lids of the reactor, can also remove the fluid centrally. Part of the fluid is then removed at (20) by passing through a control valve (24). Its flow rate is approximately equal to the flow rate of the fluid fed at (4). The rest of the fluid is treated, for example dried using a condenser and/or heated, at (19), then recycled (23) via the opposite end of the distributor (5). It should be observed that in the arrangement described above, the fluid may be recycled on average several times before being removed, if the recycled fluid flow rate (23) is several times higher than the feed flow rate (4) and hence also than the removal flow rate (20), but, due to its mixing in the fan (18), a small fraction of the fluid will be removed upon its first passage through the reactor. This can be avoided by using a second fan, (18.1), as shown in the diagram in FIG. 1.

The solid particles (25) are introduced into the reactor via the tube (26) by appropriate means and are transferred from one chamber to the next via the passages (27). The particles will first fill the first cylindrical chamber, Z1, until the level of the surface (9) of the fluidized bed reaches the level of the first passage (27). The particles can then begin to fill the second cylindrical chamber, and so on and so forth, until the time when the level of the last cylindrical chamber, Z5, reaches the level of the outlet opening of the particles (29) via the tube (30) enabling them to leave the reactor.

However, since the fluid preferentially passes through the zones containing little or no solid particles, secondary passages (27.1) must be provided, localized against the side wall of the reactor to permit progressive and more or less uniform filling of all the cylindrical chambers to avoid excessive differences in fluid flow rates in the injection slits preventing the transfer of energy necessary to rotate the solid particles in the zones being filled.

The transfer rate depends on the rotational velocity of the solid particles, the dimensions of the passages, and their profile, and differences in level of the fluidized bed surface from one chamber to the next. The latter may be accentuated or decreased by tilting the reactor.

The particles are rotated by the transfer of momentum from the fluid to the particles, to compensate for energy losses due to turbulence, friction and their transfers in the reactor, and from one chamber to the other. This momentum may be increased by placing side deflectors (not shown in the figure) appropriately profiled facing the injectors. The energy losses can be minimized by appropriately designing the internal aerodynamics of the cylindrical chambers.

In case of malfunction, the reactor can be drained via openings arranged in the bottom of each zone and a particle filter or separator can be installed upstream of the fan (18) or of the outlet (20) to avoid sending solid particles downstream of the installation.

The central openings of the hollow disks can be connected by central deflectors, like those (38) shown in FIG. 2 and their inlets may be located in the upper part of the reactor to minimize the risk of particle suction, particularly during accidental shutdowns.

FIG. 10 shows a cross section through a hollow disk along plane AA* in FIG. 9, for a reactor having two distributors and two collectors and forming with them a compact and easily transportable assembly designed to be easily disassembled. The figure shows the section (1) of the side wall of the reactor, the section (5) of two distributors, of their longitudinal slits (69), perpendicular to the plane of the figure, and of plates (70) for injecting the fluid (4) via the slits (7) passing through the reactor wall longitudinally (perpendicular to the plane of the figure), dividing it into two half-cylinders. They are preferably arranged at approximately the same height on each side of the reactor, so that the fluid flow rate through them is not affected by differences in hydrostatic pressure in the fluidized bed. They are framed by the plates (73), which are welded or prolong the side wall (1) of the reactor and which are connected, removably, to the plates (70) of the distributors (5) by fasteners (74). Their spacing is maintained by inserts (75) placed uniformly along these longitudinal slits and appropriately profiled to minimize their resistance to the flow of the fluid injected into the reactor. This device makes it possible to open the reactor by lifting its upper part.

The enlargement (12) of the hollow disk around its central opening is bounded by two circles (76), in thin lines, and the two enlargements (15) at the periphery of the disk, around its side openings, are bounded by the curves (77), in thin lines. Since the interior of the hollow disk is visible, one can see the section (78) of beams connecting its two parallel walls to maintain the spacing thereof, to increase the overall stiffness and to guide, to the openings arranged in its side wall (79), the fluid (80) which rotates rapidly when it enters the hollow disk.

The fluid (13) then leaves the hollow disk and enters the two collectors of section (17) by passing through the nozzles of which one side (16) is shown, and whereof one end (81), in a thin line, is welded to the collector (17) and whereof the other end, which penetrates into the transverse slit of the reactor, is welded to the side wall of the reactor and penetrates into the hollow disk through slits in its side wall (79). The circular end (82) of the nozzle (16) bears against the lower wall of the hollow disk and the sides of the nozzles, whereof the sections (83) are shown, are bent at their end (84) to facilitate their insertion into the openings of the side wall of the hollow disk, during the assembly of the reactor. Triangular beams (85) connect the opposite walls of the nozzles to increase their stiffness, and their appropriately profiled ends (86) penetrate into the hollow disk to guide these nozzles inside the disk during the assembly of the two parts of the reactor. The ends (82) and (84) of the nozzles (16) have dimensions suitable for fitting them easily and sufficiently tightly into the side openings of the hollow disks.

The passages for transferring the particles from one zone of the reactor to the other through the hollow disk, are arranged, for example, along the edges of the hollow disk, (27.1), and closer to its center (27.2). They are bounded by the walls (87) perpendicular to the plane of the figure and the inclined walls (52) which guide the solid particles traveling in the direction (89) from the zone on one side of the disk to the zone on the other side. If a transfer of the solid particles in both directions is desirable to obtain a reflux, for example of the heaviest particles, certain passages, for example close to the reactor wall, may be inclined in the opposite direction.

FIG. 11 is an enlargement of the fluid injection device shown in FIGS. 9 and 10. It shows, cross-hatched, a part of the section (1) of the side wall of the reactor, of the distributor (5), of the plates (70) and (73) connecting the longitudinal slit (7), perpendicular to the plane of the figure, in the reactor wall to the longitudinal slit (69) of the distributor (5) of the fluid (4), and in thin lines, the fastener (74) used to assemble the lower part of the reactor, to the left of the figure, with its upper part, to the right, and the section of the insert (75) for spacing the plates (73) whereof one is the prolongation of the wall (1) of the upper part of the reactor, to the right, and the other is welded to the lower part of the reactor, to the left. The side wall (79) of the hollow disk and a passage (27.1), along the side edge of the hollow disk, bounded by a side wall (87) and inclined walls (88) which guide the stream of particles (89) from the zone below the hollow disk to the zone above the hollow disk, are also visible in this figure.

The sections of side deflectors (32), like those described in FIG. 2, are not shown. They could coincide with or be offset from the section (87) of the side wall of a passage and be prolonged beyond the passage, as required.

FIG. 12 shows a cross section, along plane BB* perpendicular to FIG. 10, of the nozzle connecting a hollow disk to a collector. It shows the outer surface of the collector (17), the inner surface of the side (79) of a hollow disk and the section (3) of its two parallel walls, the two circular ends (82) and the ends (84) of the triangular side edges of the nozzle, bent and profiled for insertion into the opening (14), arranged in the side wall (79) of the hollow disk between its walls (3), the triangular beams (85) with their appropriately profiled ends (86) to facilitate the fitting of the nozzle into the opening of the hollow disk, and finally, the upper and lower wall (16) of the nozzle which intersects the collector (17) along the weld lines (81)

To provide orders of magnitude, these various methods can be illustrated by quantified examples. However, since the rotational velocities of the particles depend on a set of factors such as turbulence and viscosity of the fluidized bed, which depend on the type of solid particles and the aerodynamics in the cylindrical chambers, the examples below are only given for information.

### FIRST EXAMPLE

### Catalytic Bimodal Copolymerization of Ethylene and Hexene

For information, an industrial scale unit, according to the diagram in FIG. 7, can, for example, have cylindrical chambers 3 m in diameter and 1.8 m in height. If the ethylene pressure is about 25 bar and if the particle concentration in the fluidized bed is about 35%, the ratio of the density of the fluidized bed to that of the fluid is about 11.

Central openings of the hollow disks 0.8 m in diameter are suitable for easily removing a recycled ethylene flow rate of 5 m³/sec per cylindrical chamber, or about 500 tonnes per hour. If the polymer particles are transferred from one chamber to the other at a rate of 125 liters per second, or about 150 tonnes per hour and slightly more if the profile of the passages is designed to increase the particle concentration therein to reduce the transfers of undesirable fluids from one chamber to the other, an average fluid injection rate of about 20 m/sec and an efficient transfer of momentum from the fluid to the polymer particles can be suitable for rotating them at an average velocity of more than 6 m/s, which is sufficient to obtain a vertical rotating fluidized bed.

If the thickness of the fluidized bed at the tops of the cylindrical chambers is about 30 cm, the thickness at their bases may be about 0.9 m, giving a fluidized bed volume of nearly 7 m³ per cylindrical chamber, or about 2.3 tonnes of polyethylene. The use of helical turns or other appropriate means serves to increase the thickness at the tops of the chambers, while reducing it at their bases, thereby possibly permitting a fluidized bed volume of 7.5 m³ or 2.5 tonnes of polyethylene, while reducing the differences in pressures, velocities and residence times of the fluid in the fluidized bed between their bases and their tops.

The average residence time of the polymer particles in each cylindrical chamber is about 1 minute and that of the fluid in the fluidized bed is 1.5 seconds. If the reactor comprises 10 cylindrical chambers, which may be grouped in two or more sets having separate recycle circuits, to obtain a bimodal or multimodal polymer particle composition, the total volume of recycled fluid is 50 m³/sec, or about 5400 tonnes per hour, making it possible, without using refrigerants, to cool a production of at least 50 tonnes of polymer per hour with an average particle residence time of 30 minutes, permitting them about three complete cycles on average, thereby ensuring a reasonable uniformity of the polymer particles, while limiting the transfers of undesirable fluids between the various parts of the reactor. If priority is assigned to the uniformity of the polymer particles, the quantity of polymer particles transferred from one chamber to the other can be increased, by increasing the dimensions of the passages, thereby also increasing the quantity of undesirable fluids transferred from one set of chambers to the other, and thereby possibly decreasing their differentiation.

The volume of ethylene fed to the reactor being about 0.5 m³/sec, or about 6 times the volume of fluid transferred with the particles from one chamber to the other and hence also in the purification column (61), it is easy to purge the particles of this fluid containing hexene by using part of this ethylene in this column, owing to the possibility of having a lower hexene concentration in the upper cylindrical chamber, if the hexene is only sprayed in the lower cylindrical chambers of the upper set.

If the lower set of cylindrical chambers contains a high hydrogen concentration to decrease therein the molecular weight of the high density polyethylene produced therein, a small quantity of this hydrogen is transferred to the upper set(s) of the reactor at the same time as the polymer particles. To prevent its concentration therein from being too high, it can be controlled using a hydrogen absorber which can be inserted in the fluid recycle circuit(s) of the upper set(s).

It is the surface area of the fluidized bed of about 12 m² per chamber or 120 m² overall for an average fluidized bed thickness of about 0.6 m, and the centrifugal force, which permit such a high fluid flow rate and such a short fluid residence time in the fluidized bed. Since the cylindrical chambers are fed in parallel, the pressure difference between the inlet and outlet of the reactor is relatively small, in order to limit the energy expenditure necessary to recycle the fluid. The centrifugal force and the fluid travel direction, essentially tangential to the surface of the fluidized bed, allow a high difference in velocity between the fluid and the particles, to obtain a better heat transfer, without excessively decreasing the density of the fluidized bed.

### SECOND EXAMPLE

### Catalytic Cracking of Light Olefins

The catalytic cracking of gasoline olefins produced by catalytic crackers occurs at high temperature and low pressure, close to atmospheric pressure. It is highly endothermic, justifying operation in two successive passes with intermediate heating, requiring the flow of a considerable volume of fluid. The catalyst is progressively covered with carbon, at a rate which increases as the fluid to crack is heavier, justifying a catalyst circulation with continuous regeneration. The average cycle time between two regenerations depends on the operating conditions. It may be between less than one hour to several hours.

For example, for information and to provide the orders of magnitude, an industrial reactor may have cylindrical chambers 1.6 m in diameter and 1.5 m high. If the ratio of the fluidized bed density to the fluid density is 150, a recycled fluid flow rate of 2.4 m³/sec, injected at an average velocity of 50 m/sec, can rotate the catalyst particles at a rotational velocity above 4 m/sec, sufficient to obtain a vertical rotating fluidized bed. Since the differences in rotational velocity of the particles, in the pressures and thicknesses of the fluidized bed between the top and bottom of the chambers may be fairly high, it is desirable to equip them with upward helical turns or other devices for reducing them. This can help to obtain a fluidized bed of a thickness of between 20 and 40 cm, with a volume of about 1.7 m³ and a surface area of 5 m² per chamber, with an average fluid residence time of 0.7 seconds in the fluidized bed.

If the reactor comprises two sets in series of four cylindrical chambers each, giving it a height of more than 12 meters on account of the thickness of the hollow disks necessary for removing the fluids, it can crack about 200 tonnes per hour, if the density of the heated fluid is 6 g/liter.

The pressure difference between the inlet and outlet of each set of cylindrical chambers, necessary to offset the hydrodynamic pressure of the fluidized bed and inject the fluid at the desired velocities, may be less than a quarter of the atmospheric pressure. If the pressure drop in the heating oven is sufficiently low, if the two parts of the reactor are fed in series, the pressure difference between these two parts may be less than 50% of the atmospheric pressure, to be compared with the hydrostatic pressure of the fluidized bed in the recycle column (61), which may be close to atmospheric pressure for a height of 11 m which is sufficient to recycle the regenerated catalyst particles.

One of the advantages of this series configuration is the lower fluid pressure in the outgoing reactor, thereby favoring its conversion. This configuration also serves to use more than two parts of the reactor in series, thereby improving the conversion, without a very high extra cost, on account of the short distances possible between the furnaces and the reactor and the lack of any need for an additional compressor.

### THIRD EXAMPLE

### Horizontal Grain Dryer

To provide the orders of magnitude, a horizontal reactor as shown in FIGS. 9 to 12, forming with these accessories an assembly of the size of an easily transportable container, may have a diameter of 1.8 m and can be divided into 6 cylindrical chambers 0.5 m wide. The wet grains (25) are introduced via the tube (26) into zone Z1. They are heated and dried by the recycled air, which is heated by the heat exchanger (19) and optionally dried, if necessary, by a condenser, not shown. The grains are transferred from one cylindrical chamber to the other up to the final chamber, Z6, where they are cooled by the cold air (6) which they preheat completing their drying before exiting (29) via the tube (30). The air is heated, dried and recycled to the other zones, a number of times equal to the ratio of the total flow rate of the fan to the flow rate of the air removed at (20).

Since the fluid travels in the fluidized bed essentially parallel to the surface of the fluidized bed and the centrifugal force allows a relatively high radial velocity perpendicular to this surface, the difference in velocity between the air and the grains and the air flow rate may be relatively high, thereby reducing the time necessary for drying. Moreover, since the grains are cooled by cold air before leaving the reactor and their residence time in the reactor is relatively short, they may be heated to slightly higher temperatures than in a conventional dryer. Furthermore, since the humid air is slightly cooled by the grains which it preheats before leaving the reactor, the use of the heat is highly effective. This effectiveness can be improved by using a second, smaller fan, which directly removes the air leaving the first cylindrical chamber, which has served to preheat the grains and which can be isolated by a separation in the first hollow disk, without being mixed with the air issuing from the other cylindrical chambers. Moreover, small secondary passages, (27.1), along the side wall of the reactor, can ensure a preferential transfer of the heaviest grains, which are therefore the most difficult to dry, in the reverse direction, in order to increase their residence time in the reactor.

If, for example, the fluidized bed containing the grains in suspension has a bulk density of 300 grams per liter, the ratio of this density to the ambient air is about 230, requiring a very high air flow rate and injection velocity. For example, an air flow rate of 2 m³/sec per chamber, or more than 9 tonnes per hour per chamber, injected at about 40 m/sec, and an efficient transfer of momentum from the air to the grains, can produce rotational velocities of the grains of over 6 m/sec, giving a thickness difference of less than 12 cm between the top and the bottom of a fluidized bed with an average thickness of 30 cm.

The total air flow rate of 12 m³/sec can be supplied by a fan in two distributors 0.65 m in diameter and removed by two collectors 0.7 m in diameter, the central openings of the hollow disks possibly being smaller than 0.6 m in diameter. This serves to contain the assembly formed by the reactor, its distributors and collectors, in a 2.5 m sided square, corresponding to the size of a standard container.

The volume of the fluidized bed is about 700 liters per chamber, or 4.2 m³ overall, for a surface area of over 11 m². If the grains are transferred from one chamber to the other at 20 liters per second, or about 20 tonnes per hour, their average residence time in the dryer is about 3.5 minutes. Their degree of drying depends on the moisture content and temperature of the air which can be heated, inter alia, by the cooling of the fan motor, and can pass through a condenser, but in general, it is more rapid than in an ordinary dryer, owing to the large difference in velocity between the air and the grains, obtained because of their tangential direction and the centrifugal force.

In case of unscheduled shutdown, it is necessary to provide a cyclone and/or filter to prevent part of the grains from being entrained by the fan and removed in the atmosphere, and openings in the bottom of each zone in order to drain the reactor before restart.

The capacity can be doubled by doubling the length of the reactor and by using an additional fan on

I claim:

46. A rotating fluidized bed device comprising:
a cylindrical reactor;
a device for feeding solid particles to said reactor;
a device for feeding fluids into fluidized beds along a cylindrical wall of said reactor in directions tangential to said cylindrical wall and perpendicular to a central axis of said reactor, wherein said fluids or fluid mixture puts the solid particles in suspension in the fluidized beds, and wherein said fluids or fluid mixture is fed to cause the fluidized beds to rotate with a velocity that produces a centrifugal force thrusting said solid particles toward said cylindrical wall, and wherein said fluids are gaseous or liquid, or a mixture thereof;
wherein the reactor is divided into a succession of cylindrical chambers by a succession of hollow disks fixed against a side wall of the reactor, and wherein the hollow disks have openings to interconnect the chambers and allow passage of the fluid and the solid particles in suspension in the fluid through each chamber;
a device for removing said fluids or fluid mixture from the central axis of said reactor, wherein each said hollow disks further has at least one side opening connected to at least one collector outside the reactor for removing said fluids through said hollow disks and for regularizing outlet pressures of said cylindrical chambers; and
a device for removing said solid particles in suspension from said rotating fluidized beds and from said reactor.

47. (canceled)

48. The device of claim 46, wherein the hollow disks have profiled passages to enable the solid particles suspended in the fluid to pass from one cylindrical chamber to the another.

49. (canceled)

50. The device of claim 46, wherein said device for feeding said fluid or fluid mixture uniformly distributes the fluids into the beds and is further comprised of side deflectors placed close to the fluid injectors for mixing said fluid or fluid mixture with part of said solid particles rotating in said cylindrical chambers and for accelerating said particles in the spaces bounded by said side deflectors, said side deflectors being profiled to enable said fluid to transfer energy to said solid particles before leaving said bounded spaces and to enable said solid particles to transfer an acquired momentum to the other said solid particles rotating in said cylindrical chambers after said particles leave said bounded spaces.

51. The device of claim 46, wherein said openings of said hollow disks have one or more deflectors that pass longitudinally through said cylindrical chambers and which have curvatures bounding one or more access slits through which said fluid or fluid mixture is moved out toward said central openings, said curvatures and said access slits being arranged to reduce a probability of said solid particles entering said openings of said hollow disks.

52. The device of claim 46, wherein at least one of said hollow disks contains one or more separating partitions for separating said fluid or fluid mixture which enters said hollow disk and which exits from said cylindrical chambers separated by said hollow disk.

53. The device of claim 46, wherein at least one of said hollow disks permits a passage of an injector capable of spraying fine droplets of a secondary fluid on the surface of at least one said rotating fluidized bed of at least one of said cylindrical chambers, and wherein at least one of the other said fluids is a gas.

54. (canceled)

55. The device of claim 46, further comprised of a device for recycling said removed fluid or fluid mixture after suitable treatment to said cylindrical chambers.

56. The device of claim 46, wherein said device for feeding said solid particles to said cylindrical chamber is located at one end of said reactor and wherein said device for removing said solid particles from said cylindrical chamber is located at another end of said reactor.

57. (canceled)

58. The device of claim 46, wherein said device for removing said solid particles from one said cylindrical chamber is servocontrolled by a device that detects the surface of said rotating fluidized bed of said chamber, and wherein said servocontrol is capable of maintaining said surface at a desired distance from the cylindrical wall of said chamber.

59. The device of claim 46, wherein said passages are profiled to facilitate a transfer of said solid particles from one said cylindrical chamber at a top of the reactor to the other chambers toward an end of said reactor, wherein said passages are located at a desired distance from said central openings of said hollow disks in order to stabilize said surfaces of said rotating fluidized beds therein, and wherein a flow rate of the particles towards said end increases or decreases according to whether said passages are more or less immersed in said rotating fluidized beds.

60. The device of claim 46, wherein said device further comprises passages located along said cylindrical wall of said reactor that are profiled to facilitate a transfer of said solid particles from one said cylindrical chamber to another in a direction suitable for progressively filling all of said cylindrical chambers of said reactor or emptying said solid particles from all of said cylindrical chambers of said reactor.

61. The device of claim 46, wherein said device further comprises secondary passages which are located along said cylindrical wall of said reactor and which are profiled to facilitate a transfer of said solid particles from one said cylindrical chamber to the other in a direction opposite to that of the other said passages in order to provide a reflux of the solid particles.

62. (canceled)

63. The device of claim 46, wherein said device for feeding said fluid or fluid mixture to at least one of said cylindrical chambers is servocontrolled by a device that detects a surface of said rotating fluidized bed of said cylindrical chamber, said servocontrol suitable for maintaining said surface of said bed at a desired distance from a side wall of each said cylindrical chamber.

64. The device of claim 63, wherein said device for feeding said fluid or fluid mixture comprises longitudinal slits through the said side wall of each said cylindrical chamber parallel to the axis of symmetry of said reactor, said longitudinal slits being connected to at least one fluid distributor outside said reactor that is suitable for regularizing the inlet velocities of said fluid or fluid mixture injected into said reactor via said slits.

65. The device of claim 64, characterized in that said longitudinal slits pass through said side wall from one end of said reactor to the other to dividing said cylindrical wall of said reactor into at least two cylinder fractions.

66. The device of claim 46, wherein said device for removing said fluid or fluid mixture comprises transverse slits, perpendicular to the axis of symmetry of said reactor that passing through said cylindrical wall along said side openings of said hollow disks, said transversal slits being connected to at least one fluid collector outside said reactor that is suitable for regularizing an outlet pressure of said fluid or fluid mixture removed from said reactor via said transfer slits.

67. The device of claim 66, wherein said removal device further comprises two said distributors and two said collectors each distributor and collector comprised of a tube running along said cylindrical wall of said reactor, said four tubes forming an assembly with said reactor.

68. The device of claim 67, wherein said assembly is compact, removable, and transportable.

69. The device of claim 46, wherein said reactor is operated in a horizontal position.

70. The device of claim 69, wherein said reactor is inclinable to increase or decrease a transfer of said solid particles through said passages toward said removal device, without any significant change in a volume of said fluidized bed.

71. The device of claim 69, wherein said access slits are arranged in an upper half of said reactor to decrease a probability of said solid particles entering into said hollow disks during shutdowns.

72. (canceled)

73. The device of claim 46, wherein the reactor is vertical.

74. The device of claim 73, wherein the walls of said cylindrical chambers are equipped with transverse fins or helical turns that enable said solid particles to use part of their rotational kinetic energy to rise along the fins or turns, in order to reduce differences in pressure and thicknesses of said rotating fluidized beds between a top and a bottom of said cylindrical chambers, and wherein said device further comprises a transfer column or tube outside said reactor for recycling said solid particles removed from one said cylindrical chamber at one end of said reactor to said cylindrical chamber located at the other end of said reactor.

75. The device of claim 46, wherein said device further comprises at least two sets of successive cylindrical chambers and at least one said passage for transferring said solid particles from one said set to the other said set, and wherein said devices for feeding and removing said fluid or fluid mixture are suitable for feeding said fluid or fluid mixture removed from one of said sets to the other said set.

76. The device of claim 46, wherein said device further comprises at least two sets of said successions of said cylindrical chambers and at least one said passage for transferring said solid particles from one said set to the other said set, and wherein said devices for feeding and removing said fluid or fluid mixture are suitable for separately removing said fluid or fluid mixture from each of said sets and for recycling it to the same said set.

77. A method of treating solid particles in a rotating fluidized bed, using the device of claim 69.

78. A method of polymerizing solid particles in suspension in a rotating fluidized bed using the device of claim 77, wherein at least one of said fluids contains alpha-olefins.

79. A method for catalytic conversion of a fluid or fluid mixture passing through a rotating fluidized bed using the device of claim 46, wherein the solid particles are comprised of a catalyst.

80. The method of claim 79, wherein said fluid or fluid mixture contains light olefins and wherein the catalytic conversion causes a change of a molecular weight distribution of said light olefins.

81. The method of claim 79, wherein said fluid or fluid mixture contains ethylbenzene and wherein said catalytic conversion causes dehydrogenation of the ethylbenzene to thereby convert the ethylbenzene to styrene.

82. The method of claim 81, wherein said solid particles contain components which can react with hydrogen produced by said dehydrogenation in order to reduce the hydrogen concentration in said fluid or fluid mixture, and wherein said components are regenerable outside the reactor.

83. A method of drying or extracting volatile compounds from said solid particles in a rotating fluidized bed, using the device of claim 46.

84. A method of impregnating said solid particles with said secondary fluid in a rotating fluidized bed, using the device of claim 53.

85. The method of claim 83, wherein said solid particles are of an agricultural origin.

86. The method of claim 84, wherein said solid particles are of an agricultural origin.

87. A method of using the reactor of claim 46, wherein said reactor is operated in a horizontal position, wherein a fluid or fluid mixture is injected into a horizontal cylindrical reactor at a velocity and at a flow rate that gives said solid particles an average rotational velocity that is higher than a square root of a product of a reactor diameter and g, which is the gravitational acceleration.

88. A method of using the reactor of claim 46, wherein said reactor is operated in a vertical position, and wherein a fluid or fluid mixture is injected into the vertical reactor at a velocity and at a flow rate generating a centrifugal force greater than the force of gravity in said rotating fluidized bed, said solid particles being transferred from one said cylindrical chamber to another toward a bottom of said reactor, and wherein said reactor is cylindrical.

89. A method of using the reactor of claim 46, wherein said reactor is operated in a vertical position, and wherein a fluid or fluid mixture is injected at a velocity and at a flow rate giving said solid particles an average rotational velocity higher than a velocity that said particles could acquire by falling from a top to a bottom of said cylindrical chambers, and wherein said velocity enables the particles to pass from a lower cylindrical chamber to an upper cylindrical chamber via at least one passage arranged in said hollow disk, and wherein said velocity is oriented in a direction making said solid particles rise.

90. A method of catalytically converting fluids passing through rotating fluidized beds using the reactor of claim 69, and wherein a fluid or fluid mixture is injected into a horizontal reactor at a velocity and at a flow rate giving said solid particles an average rotational velocity that is higher than a square root of a product of a reactor diameter and g, which is a gravitational acceleration, and wherein said reactor is cylindrical.

91. A method for catalytic polymerization of solid particles in suspension in rotating fluidized beds using the device of claim 75, comprising the step of injecting a fluid that regenerates the catalysts present in said solid particles into a transfer tube or column prior to recycling said solid particles to said reactor.

92. The method of claim 91, further comprised of the step of injecting a fluid that purges undesirable fluids that are entrained by said solid particles prior to recycling said solid particles to said reactor.

93. A method of catalytic conversion of fluids passing through rotating fluidized beds using the device of claim 75, comprising the step of injecting a fluid that regenerates the catalysts present in said solid particles into a transfer tube or column prior to recycling said solid particles to said reactor.

94. A method for catalytic polymerization of solid particles in suspension in rotating fluidized beds using the device of claim 76, comprising the step of recycling said fluids or fluid mixtures used for producing bimodal or multimodal polymers that contain active fluids of different compositions to at least two sets of successions of cylindrical chambers, said fluids or fluid mixtures are separately removed from said sets from one set to the other, to at least two, and wherein each set contains fluids of a different composition from the other sets.

95. A method for catalytic polymerization of solid particles in suspension in rotating fluidized beds using the device of claim 53, comprising the step of spraying fine droplets of a comonomer on the surface of said rotating fluidized bed of at least one said cylindrical chamber through an injector.

96. A method for catalytic polymerization of solid particles in suspension in rotating fluidized beds using the device of claim 53, comprising the step of using an injector to spray a liquid that cools said solid particles on the surface of said fluidized bed of at least one said cylindrical chamber.

97. A method for drying cereal grains, using the device of claim 46.
